(19) United States
(12) Patent Application Publication
(10) Pub. No.: US 2020/0210767 A1
(43) Pub. Date: Jul. 2, 2020

(54) METHOD AND SYSTEMS FOR ANALYZING MEDICAL IMAGE DATA USING MACHINE LEARNING

(71) Applicant: THE GENERAL HOSPITAL CORPORATION, Boston, MA (US)

(72) Inventors: Synho Do, Lexington, MA (US); Michael Lev, Newton, MA (US); Thomas Brady, Cambridge, MA (US)

(21) Appl. No.: 16/644,895

(22) PCT Filed: Sep. 10, 2018
(86) PCT No.: PCT/US2018/050262

§ 371 Date: Mar. 5, 2020

Related U.S. Application Data
(60) Provisional application No. 62/555,744, filed on Sep. 8, 2017.

(51) Int. Cl. G06K 9/62; G06T 11/00; G16H 15/00; G16H 50/20; G16H 40/20; G16H 30/40

(52) U.S. Cl. CPC G06K 9/6256; G06K 9/6268; G06T 11/008; G16H 15/00; G06T 2210/41; G16H 40/20; G16H 30/40; G06K 2209/21; G06K 2209/051; G16H 50/20

(57) ABSTRACT

A method and systems for analyzing medical imaging using machine learning are provided. In some aspects, the method includes using an input on the computing device to receive image data acquired from a subject, wherein the image data is in a raw data domain, applying, using the computing device, a trained machine learning algorithm to the image data, wherein the trained machine learning algorithm is configured to perform a predetermined analysis on the image data. The method also includes generating a report indicative of the predetermined analysis using the computing device.

### CROSS-REFERENCE TO RELATED APPLICATIONS

This application is based on, claims priority to, and incorporates herein by reference in their entirety U.S. Ser. No. 62/555,744 filed on Sep. 8, 2017 and entitled “Transformed domain machine learning for medical image diagnosis.”

### BACKGROUND

The present disclosure relates to machine learning, and more particularly to systems and methods for analyzing medical image data using machine learning.

Medical imaging represents a critical component in modern day medicine for detecting and treating diseases. Often with the help of various automated or semi-automated computational tools, clinicians utilize medical images to identify abnormal tissues, critical organs or structures at risk, as well as quantify other important findings. Among the various computational techniques being used, artificial intelligence has become increasingly popular. In particular, increasing computer capabilities and the accumulation of large, well-annotated datasets have allowed machine learning to develop rapidly, and open the door to numerous applications. For instance, machine learning has been applied to medical image analysis in order to improve diagnostic accuracy, and reduce delays in diagnosis and treatment.

Conventionally, machine learning (e.g. deep learning) algorithms utilize test images to learn features or properties that can be used to make predictions on unknown images. For instance, common machine learning algorithms are used to identify specific tissues (e.g. benign or malignant tissues) in a patient medical images. However, conventional algorithms require analysis of images reconstructed from raw signals. Such image reconstruction is often problematic because it is computationally expensive, and needs several correction steps. This is because the reconstruction process often introduces artifacts and distortions, which require correction to make the images suitable for review. In addition, a large number of images are needed to accurately estimate the unknowns corresponding to parameters and annotations. Often, large numbers of images are not accessible.

Therefore, there is a need for improved technologies capable of efficient and accurate image analysis.

### SUMMARY OF THE INVENTION

The present invention overcomes the aforementioned drawbacks by providing a method and systems for analyzing medical image data using machine learning. The foregoing and other aspects and advantages of the invention will appear from the following description.

In accordance with one aspect of the present disclosure, a method for analyzing image data using machine learning is provided. The method includes using an input on the computing device to receive image data acquired from a subject, wherein the image data is in a raw data domain, applying, using the computing device, a trained machine learning algorithm to the image data, wherein the trained machine learning algorithm is configured to perform a predetermined analysis on the image data. The method also includes generating a report indicative of the predetermined analysis using the computing device.

In accordance with another aspect of the present disclosure, a system for analyzing image data using machine learning is provided. The system includes an input in communication with an image data source and configured to receive image data therefrom and at least one processing unit. The at least one processing unit is configured to receive, from the input ,image data acquired from a subject, and apply a trained machine learning algorithm to the image data, wherein the trained machine learning algorithm is configured to perform a predetermined analysis on the image data. The at least one processing unit is also configured to generate a report indicative of the predetermined analysis. The system further includes an output configured to provide the report.

In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1. is an example system for analyzing image data, in accordance with aspects of the present disclosure.

FIG. 2 is another example system for analyzing image data, in accordance with aspects of the present disclosure.

FIG. 3 shows a flowchart setting forth steps of a process, in accordance with aspects of the present disclosure.

FIG. 4 are images of corresponding body regions shown in an image domain and sinogram domain.

FIG. 5 is a graphical representation showing an example process for generating sparse-view sinograms from linear attenuation coefficient or computed tomography (CT) images, in accordance with aspects of the present disclosure.

FIG. 6 shows example images for three different types of sonograms, and corresponding reconstructed images illustrated with and without brain window setting applied.

FIG. 7 is a graphical illustration showing an example network architecture, in accordance with aspects of the present disclosure.

FIG. 8A is a graph showing performance of the present approach for body part recognition, in accordance with aspects of the present disclosure.

FIG. 8B is another graph showing the performance of the present approach for intracranial hemorrhage detection, in accordance with aspects of the present disclosure.

### DETAILED DESCRIPTION

The present disclosure introduces a novel approach for analyzing medical image data using machine learning. In particular, it is for the first time recognized herein that machine learning need not rely on reconstructed images for analysis in the same way as human experts do. Rather, machine learning can operate directly on raw data without need for image reconstruction. This is because all information present in reconstructed images is already encoded in the raw data. Therefore, computational models could potentially decode such information by directly analyzing the raw data.

As appreciated from the description that follows, the present method and systems overcome problems and provide advantages over prior techniques, and thereby represent a significant improvement to the field of medical imaging. For instance, by virtue of the ability to produce accurate results with limited or sparse data sets, the present approach allows for radiation doses below current limits imposed by current low-dose imaging practices. In addition, the present technology can be used with simple imaging systems, such as sparse view CT scanners, stationary CT scanners, since only a few sources and detectors are needed to acquire limited or sparse data, and image reconstruction is not required.

The present technology can also be used to improve the detection performance of some current imaging systems. For instance, low-field magnetic resonance imaging systems may be improved because unstable Inverse Fast Fourier Transforms (IFFTs) or iterative image reconstruction methods would not be required. Also, the present approach makes material decomposition possible in the sinogram domain for systems that operate using photon-counting detectors.

Furthermore, the present approach allows for new imaging capabilities. For instance, given that beam forming is not required to construct ultrasound images, a large region of interest (ROI) ultrasound system could be built using the present technology. In addition, an ultrasonic CT system could be developed, since only RF signals would be needed to detect and classify normal, abnormal, or foreign materials being imaged.

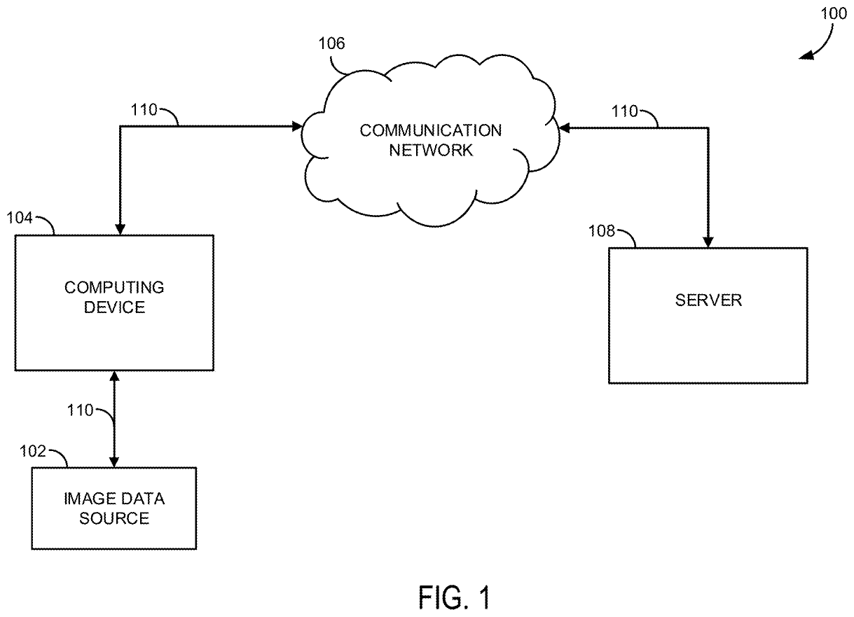

Referring now to FIG. 1, an example of a system 100, in accordance with aspects of the present disclosure, is shown. As shown, the system 100 may include an image data source 102, and a computing device 104 that is configured to retrieve and process various types of image data from an image data source 102. In some configurations, the system 100 may also include a communication network 106 and a server 108.

The computing device 104 may be configured to communicate with a server 108 using the communication network 106 to exchange various data and information, including image data received or accessed from the image data source 102 and any information obtained therefrom. In addition to being configured to carry out various operational and processing steps, the computing device 104 may also be configured to analyze image data from the image data source 102 using a trained machine learning algorithm. In some implementations, the server 108 may be configured to execute at least a portion of the machine learning algorithm. In such configurations, the server 108 can exchange data and information with the computing device 104 (and/or any other suitable computing device) and provide data and information indicative of an output generated using the machine learning algorithm.

The image data source 102 may be any suitable source of image data. For instance, the image data source 102 may include an imaging system, such as a computed tomography (CT) system, an magnetic resonance (MR) system, an ultrasound (US) system, an ultrasonic CT system, a positron emission tomography (PET) system, single photon emission computed tomography (SPECT) system, or an x-ray imaging system. Additionally, or alternatively, the image data source 102 may include another computing device (e.g., a server storing image data), or a data storage location (e.g. a database, hard disk).

In some configurations, the image data source 102 can be local to the computing device 104. For example, the image data source 102 can be incorporated with the computing device 104 (e.g., the computing device 104 can be configured as part of a device for capturing and/or storing images). As another example, the image data source 102 can be connected to the computing device 104 by a cable, a direct wireless link, or other communication link. Additionally or alternatively, in some configurations, the image data source 102 can be located remotely from computing device 104, and can exchange image data, and other data and information with the computing device 104 (and/or the server 108) via a communication network (e.g., the communication network 106).

The computing device 104 and/or server 108 can be any suitable computing device, or combination of devices, that includes one or more desktop computer, laptop computer, smartphone, tablet computer, wearable computer, server computer, a virtual machine being executed by a physical computing device, and the like.

The communication network 106 can be any suitable communication network or combination of communication networks. For example, the communication network 106 can include a Wi-Fi network (which can include one or more wireless routers, one or more switches, and others components), a peer-to-peer network (e.g., a Bluetooth network), a cellular network (e.g., a 3G network, a 4G network, and others, complying with any suitable standard, such as CDMA, GSM, LTE, LTE Advanced, WiMAX, etc.), a wired network, etc. In some configurations, the communication network 106 can be a local area network, a wide area network, a public network (e.g., the Internet), a private or semi-private network (e.g., a corporate or university intranet), other suitable type of network, or any suitable combination of networks. Communications links 110 connecting the image data source 102, the computing device 104 and server 108, as shown in FIG. 1, can each be any suitable communications link or combination of communications links, such as wired links, fiber optic links, Wi-Fi links, Bluetooth links, cellular links, and so forth.

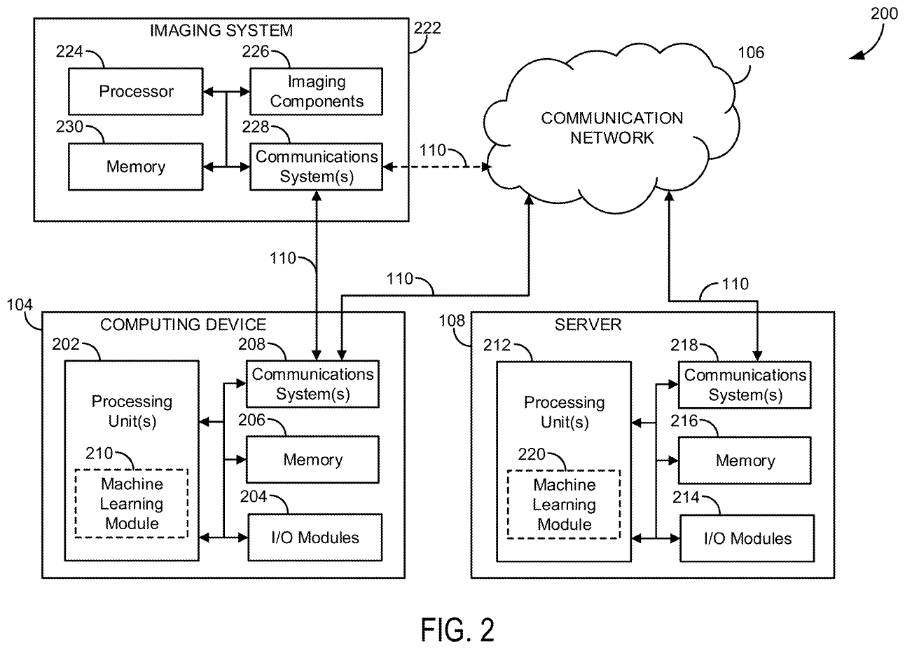

FIG. 2 shows another example of a system 200, in accordance with aspects of the present disclosure. As shown in the figure, the system 200 may include a computing device 104, a communication network 106 and a server 108. The computing device 104 may include one or more processing units 202, one or more input/output (I/O) modules 204, a memory 206, and one or more communication systems 208.

The processing unit(s) 202 of the computing device 104 can be any suitable hardware processor or combination of processors, such as a central processing unit (CPU), a graphics processing unit (GPU), and the like. In some implementations, the processing unit(s) 202 may also include a machine learning module 210 specifically configured to carry out a machine learning processing and analysis, in accordance with aspects of the present disclosure. In particular, the machine learning module 210 may be configured, by virtue specialized structure, hardwired circuitry or programming, to train and apply a machine learning model in accordance with a desired application or function. For instance, the machine learning module 210 may be configured to receive image data acquired from a number of subjects, and generate a neural network architecture (e.g. a deep neural network) that is configured to perform detection, classification or segmentation of desired tissues, structures or organs. The machine learning module 210 may then apply utilize such neural network architecture to analyze image data from a subject.

In accordance with aspects of the present disclosure, image data received by the machine learning module 210 is in a raw data format or data domain, and may include any combination of CT, MR, SPECT, PET, US and other image data types. For example, the image data may include sinogram data, k-space data, RF data, radioactivity data, and so forth. In some implementations, received image data may be pre-processed by way of being filtered, corrected for artifacts, sampled, up-sampled, down-sampled, resized, vectorized, reduced, scaled, decomposed, aggregated, integrated, interpolated, transformed, and subjected to other processing techniques known in the art. However, in accordance with aspects of the present disclosure, the image data represents data that has not been reconstructed into images. To this end, the machine learning module 210 may be configured to perform such pre-processing. Alternatively, or additionally, the processing unit(s) 202 may be configured to carry out the pre-processing, machine learning processing and analysis by executing instructions stored in the memory 206. In some implementations, the processing unit(s) 202 may be configured to apply a reconstruction process to image data obtained from a subject in order to generate images viewable by a clinician or operator.

The I/O modules 204 in FIG. 2 may include a number of input and output elements. For instance, the I/O modules 204 may include various input devices and/or sensors (e.g. a keyboard, a mouse, a touchscreen, a microphone, and the like) that can be used to receive user selections and/or operational instructions. Output elements may include various display devices, such as a computer monitor, a touchscreen, a television, and the like. The I/O modules 204 may also include various drives and receptacles, such as flash-drives, USB drives, CD/DVD drives, and other receptacles for receiving various data, information and computer-readable media.

The memory 206 can include any suitable devices that are configured to store instructions, values, data and other information. For example, the memory 210 can include magnetic media (e.g., hard disks, floppy disks), optical media (e.g., compact discs, digital video discs, Blu-ray discs), semiconductor media (e.g., random access memory (“RAM”), flash memory, solid state drives, electrically programmable read only memory (“EPROM”), electrically erasable programmable read only memory (“EEPROM”)). The memory 206 may include non-transitory computer readable media, which includes media that is not fleeting or devoid of any semblance of permanence during transmission. By contrast, transitory computer readable media includes signals on networks, in wires, conductors, optical fibers, circuits, and other media that is fleeting and devoid of any semblance of permanence during transmission.

In accordance with aspects of the present disclosure, the memory 206 may include programing or executable instructions, stored in non-transitory computer readable media, for carrying out various image data processing and machine learning, as described. The memory 206 may also have encoded thereon various computer programs and/or executable instructions for controlling operation of the computing device 104.

The communications systems 208 can include a variety of suitable hardware, firmware, and/or software for communicating information over the communication network 106 using various communication links 110, and other suitable communication networks. For example, the communications systems 208 can include one or more transceivers, one or more communication chips and/or chip sets, and so forth. In a more particular example, the communications systems 208 can include hardware, firmware and/or software that can be used to establish a Wi-Fi connection, a Bluetooth connection, a cellular connection, an Ethernet connection, and others.

As shown in FIG. 2, the system 200 may also include a server 108 may include one or more processing units 212, one or more input/output (I/O) modules 214, a memory 216, and one or more communication systems 218. Similarly to the computing device 104, elements of the server 108 may be configured to carry out various input/output, communication and processing tasks, as described. In particular, the machine learning module 220 may be optionally included in the one or more processing units 212, and configured to carry out image data pre-processing and machine learning by executing instructions programmed or hardwired therein, or stored in the memory 216. Also, the processing, analysis, input/output, communication and other tasks may be shared between the server 108 and the computing device 104.

The system 200 may further include an imaging system 222 in communication with the computing device 104. The imaging system 222 that can be any imaging machine or scanner configured to acquire image data from a subject. For example, the imaging system 222 may be a conventional MRI scanner (e.g., a 1.5 T scanner, a 3 T scanner), a high-field MRI scanner (e.g., a 7 T scanner), an open bore MRI scanner, a low-field MRI scanner (e.g. less than 1.5 T scanner), a CT system, an US scanner, a PET scanner, a SPECT scanner, and so forth. In addition, the imaging system 222 may be a sparse view CT scanner, a stationary CT scanner, an ultrasonic CT scanner, a large ROI ultrasound scanner, and so forth.

In general, the imaging system 222 may include a processor 224, various imaging components 226, one or more communications systems 228, and/or a memory 230. The processor 224 can be any suitable hardware processor or combination of processors, such as a CPU, a GPU, and the like. The processor 224 may be configured to carry out various steps, including directing the acquisition and optionally processing image data, as well as other tasks. For instance, the processor 224 can execute programming or instructions to process user input, acquire imaging signals, assemble image data, generate images, transmit and receive information and/or content (e.g., image data), receive instructions from one or more devices (e.g., a personal computer, a laptop computer, a tablet computer, a smartphone, and the like), provide output, and so forth.

The imaging components 226 can be any hardware and components suitable to generate image data corresponding to one or more imaging modalities (e.g., T1 imaging, T2 imaging, functional MR imaging, PET imaging, ultrasound imaging, CT imaging, and so on).

Note that, although not shown, the imaging system 222 can include any suitable inputs and/or outputs. For example, the imaging system 222 can include input devices and/or sensors that can be used to receive user input, such as a keyboard, a mouse, a touchscreen, a microphone, a trackpad, a trackball, hardware buttons, software buttons, a microphone and the like. As another example, the imaging system 222 can include any number of output or display devices, such as a computer monitor, a touchscreen, a television, one or more speakers, and so on.

The communications systems 226 can include any suitable hardware, firmware, and/or software for communicating data and information to the computing device 104 (and, in some embodiments, over the communication network 106 and/or any other suitable communication networks). For example, the communications systems 228 can include one or more transceivers, one or more communication chips and/or chip sets, and the like. In a more particular example, the communications systems 228 can include hardware, firmware and/or software that can be used to establish a wired connection using any suitable port and/or communication standard (e.g., VGA, DVI video, USB, RS-232, and the like), Wi-Fi connection, a Bluetooth connection, a cellular connection, an Ethernet connection, and the like.

The memory 230 can include any suitable storage device or devices that can be used to store instructions, values, image data, and the like. In some implementations, the memory 230 includes programming or instructions executable by the processor 224 to: control the imaging components 224, and/or receive image data from the imaging components 224; generate images or image data; present content (e.g., images, output, instructions, a user interface, and the like) using a display; communicate with the computing device 104 and server 108, and so forth.

The memory 230 can include any suitable volatile memory, non-volatile memory, storage, or any of a variety of other suitable combination thereof. For example, the memory 230 can include RAM, ROM, EEPROM, one or more flash drives, one or more hard disks, one or more solid state drives, one or more optical drives, and the like. In some configurations, the memory 230 can have encoded thereon programming or instructions for controlling operation of the imaging system 222.

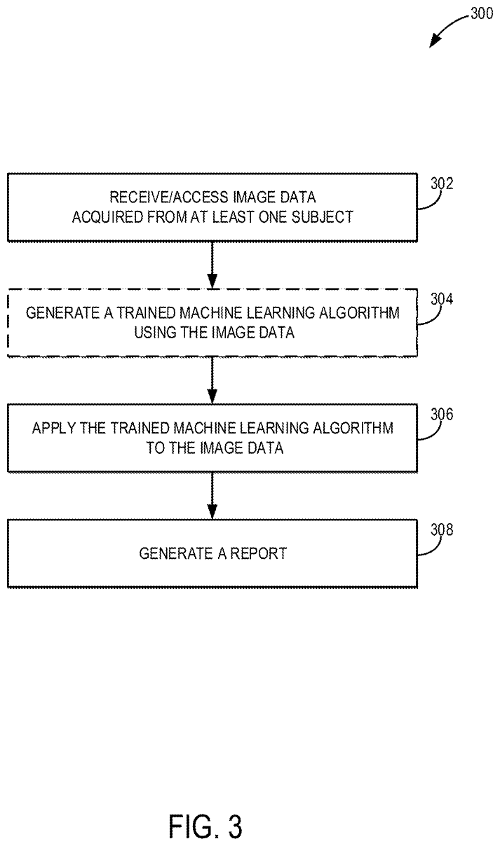

Referring now to FIG. 3, a flowchart setting forth steps of a process 300, in accordance with aspects of the present disclosure, is shown. Steps of the process 300 may be carried out using any suitable device, apparatus or system, such as systems described herein. Steps of the process 300 may be implemented as a program, firmware, software, or instructions that may be stored in non-transitory computer readable media and executed by a general-purpose, programmable computer, processor or computing device. In some implementations, steps of the process 300 may also be hardwired in an application-specific processor or dedicated module (e.g. a machine learning module).

The process 300 may begin at process block 302 with receiving or accessing image data acquired from one or more subjects. The image data may be accessed or retrieved from a database, storage server, hard disk or other location capable of storing computer-readable media. In some implementations, the image data may be acquired using one or more image systems and retrieved therefrom. Also, the image data may be in a raw data format or data domain, and include any combination of CT, MR, SPECT, PET, US, functional MR, and other image data types. For example, the image data may include sinogram data, k-space data, RF data, radioactivity data, and so forth. As such, the image data may be in a sinogram domain, a k-space domain, an RF data domain, a radioactivity data domain, and so on.

In some aspects, a pre-processing may also be carried out at process block 302 on the received or accessed image data. For instance, the image data may be filtered, corrected for artifacts, sampled, up-sampled, down-sampled, resized, vectorized, reduced, scaled, decomposed, aggregated, integrated, interpolated, transformed, and subjected to other processing techniques known in the art. However, in accordance with aspects of the present disclosure, the image data represents data that has not been reconstructed into images.

Optionally, a step of generating or updating a trained machine learning algorithm may be carried out at process block 304. By way of example, the trained machine learning algorithm may be generated or updated based on supervised, semi-supervised, unsupervised or reinforcement learning performed using training data obtained from one or more subjects. It is noted, however, that unlike conventional algorithms, the trained machine learning algorithm at process block 304 is configured to operate directly in a raw data domain, rather than an image domain.

Then, at process block 306, the trained machine learning algorithm may be applied to the image data acquired from a subject. Application of the trained machine learning algorithm can provide a variety of analyses with respect to the image data acquired from the subject. For instance, the trained machine learning algorithm may be configured to identify or detect the presence of one or more targets in the imaging data, such as specific tissues, structures or organs (e.g. benign or tumor tissues, hemorrhages, and so forth). Also, the trained machine learning algorithm may be configured to perform a classification or segmentation of the target(s) identified or detected in the image data.

In some aspects, a subset of the image data corresponding to identified or detected targets may be selected from the image data of the subject, and used separately. For example, the subset of the image data may be used in subsequent analysis (e.g. material decomposition, analysis of material properties, and so on). In some aspects, the image data, or a subset thereof, may also be used to generate one or more images, contours, graphs, tables, annotations or other visual renderings, or representations.

A report may then be generated at process block 308 based on the application of the trained machine learning algorithm. The report may be in any form, and include a variety of information. In some aspects, the report may include one or more images, contours, graphs, tables, annotations or other visual renderings, or representations highlighting or displaying information with respect to identified, classified or segmented targets. The report may be provided to a user, or relayed for further analysis to or by a suitable system or device.

As described, the present disclosure introduces a novel approach in which raw image data (e.g. sinogram data, k-space data, ultrasound RF signal data, PET data, SPECT data, and so on) may be accessed from an imaging system or a database, and directly utilized to collect unique features that can be adjusted for specific tasks. Such direct access of features from raw data removes complicated and diverse image reconstruction processes utilized in conventional approaches, and simplifies machine learning analyses.

The basis Convolutional Neural Network (CNN) may be defined by:

X^{l}=ƒ(U^{l}), with U^{l}=W^{l}X^{l-1}+B^{l} Eqn. 1

where the W is a synaptic weight, B is a bias, X is the image, l is a neural network layer. In some aspects, the output activation function ƒ(*) may be chosen to be a non-linear function, such as the logistic or sigmoidal function.

In the transformed domain,

Y=RX+n Eqn. 2

where X is the image, R is the projection of transformation of the image to raw data, Y, and n is the noise. R may be obtained through the collection of raw image data using a medical imaging scanner (e.g. a CT, MR, PET, US, and so on), and the image X can then be reconstructed using an image reconstruction process. Such process can be formulated as an inverse problem, as follows

X≅R⁻¹Y. Eqn. 3

However, rather than applying an image reconstruction process, R⁻¹, as common in conventional practice, in accordance with aspects the present disclosure, the raw data domain, Y, may be directly used. Specifically,

Y^{l}=ƒ(V^{l}), with V^{l}=ŴY^{l-1}+B^{l} Eqn. 4

As demonstrated below, applying Eqn. 4 in a machine learning algorithm can produce accurate, simple and more stable results compared with conventional approaches.

A feasibility study, described below, demonstrates features and advantages of the present approach to identify human anatomy and detect pathology using computed tomography (CT) projection data, or sinograms. This study is illustrative of the present approach, and should in no way be interpreted to limit the present invention.

### EXAMPLE

A customized convolutional neural network (CNN), hereafter referred to as SinoNet, optimized for analyzing sinograms, was developed for body part recognition and intracranial hemorrhage (ICH) detection. As appreciated from results described below, the present approach provides superior results compared to conventional CNN architectures that rely on reconstructed CT images.

In this study, and with IRB approval, two hundred whole body CT scans and 720 non-contrast head CT scans were retrieved from an institutional picture archiving and communication system (PACS) for body part recognition and ICH detection, respectively. For body part recognition, sixteen different body regions were annotated by a physician on axial CT slices. For ICH detection, the axial slices were annotated by five board-certified radiologists for presence of ICH. A 2D parallel-beam Radon transform was performed on the retrieved CT images to generate simulated sinograms with 360 projection views over 180 degrees and 729 detectors (Sino360×729). Sino360×729 was then uniformly subsampled in vertical direction (projection views) and averaged in horizontal direction (detectors) by factors of 3 and 9 to create sinograms with 120 projection views and 240 detectors (Sino120×240) and sinograms with 40 projection views and 80 detectors (Sino40×80), respectively. Furthermore, sparser sinograms were reconstructed to obtain corresponding reconstruction images to be used as a comparison with model performance using the sinograms.

TABLE 1
Distribution of training, validation, and test
dataset for body part recognition.

| | Train | Validation | Test |
|---|---|---|---|
| #Cases | 140 (70F, 70M) | 30 (15F, 15M) | 30 (15F, 15M) |
| #Images | 39,472 | 8,383 | 8,479 |
| H1 (Head) | 1,980 | 483 | 435 |
| H2 (Eye lens) | 878 | 189 | 188 |
| H3 (Nose) | 1,449 | 309 | 323 |
| H4 (Salivary gland) | 1,803 | 361 | 349 |
| H5 (Thyroid) | 1,508 | 312 | 333 |
| T1 (Upper lung) | 1,632 | 345 | 392 |
| T2 (Thymus) | 3,213 | 727 | 672 |
| T3 (Heart) | 3,360 | 707 | 762 |
| T4 (Chest) | 4,647 | 914 | 935 |
| T5 (Upper abdomen) | 4,943 | 1,008 | 1,103 |
| T6 (Lower abdomen) | 1,736 | 342 | 368 |
| T7 (Upper pelvis) | 2,524 | 617 | 545 |
| T8 (Lower pelvis) | 2,230 | 563 | 422 |
| T9 (Bladder) | 3,144 | 609 | 766 |
| L1 (Upper leg) | 2,607 | 563 | 532 |
| L2 (Lower leg) | 1,818 | 334 | 354 |

### Methods

### Data Collection and Annotation

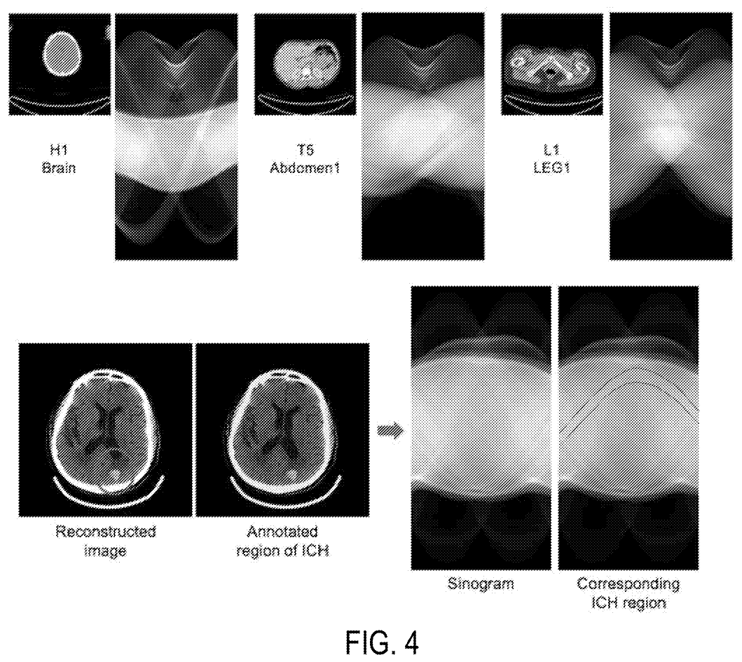

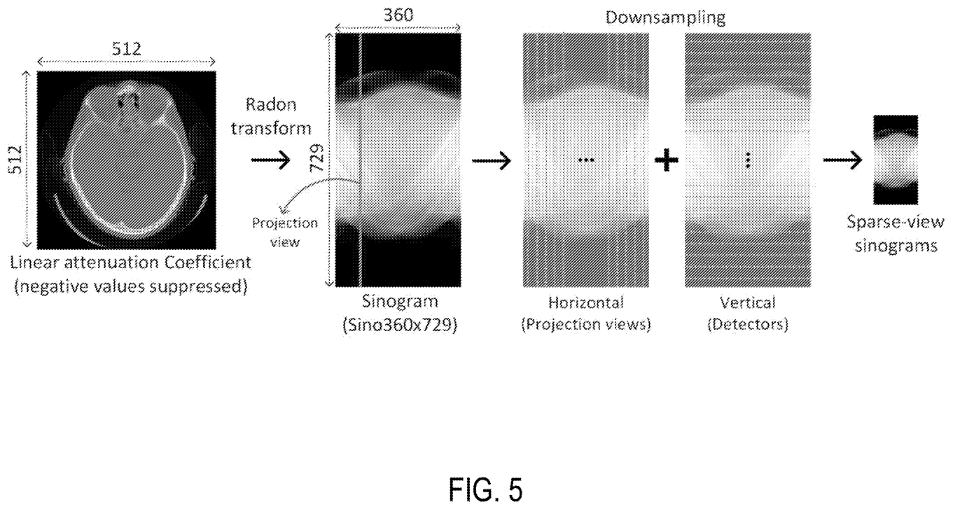

For human anatomy recognition, a total of 200 contrast-enhanced PET/CT examinations (performed from May 2012 to July 2012) of head, neck, chest, abdomen, and pelvis for 100 female and 100 male patients were retrieved from an institutional Picture Archiving and Communication System (PACS). These cases included 56,334 axial slices which were labeled as one of sixteen body regions by a radiologist (blinded for review, and having 5 years experience). Approximately 70% of the total data was randomly selected as a training dataset for model development, 15% was selected for a validation dataset for hyperparameter tuning and model selection, and 15% was selected as a test dataset for performance evaluation (Table 1). By way of example, FIG. 4 shows reconstructed and annotated CT images for different body regions, and corresponding annotated regions in the sinogram domain.

For the intracranial hemorrhage (ICH) detection dataset, patients who underwent 5-mm non-contrast head CT examinations for indication of ICH were identified from June 2013 through July 2017 from our PACS. This dataset included 201 cases without ICH and 519 cases with ICH which were randomly split into training, validation, and test datasets (Table 2). Every 2D 5-mm thick axial slice (3,151 slices without ICH and 2,895 slices with ICH) was annotated by five US subspecialty board-certified neuroradiologists (blinded for review, 9 to 34 years experience) according to presence of ICH.

TABLE 2
Distribution of training, validation, and test dataset for ICH detection.

| | Train | | Validation | | Test | |
|---|---|---|---|---|---|---|
| | #Cases | #Images | #Cases | #Images | #Cases | #Images |
| No ICH | 141 | 2,202 | 30 | 474 | 30 | 475 |
| ICH | 337 | 1,915 | 91 | 490 | 91 | 475 |
| Total | 478 | 4,117 | 121 | 964 | 121 | 950 |

### Sinogram Generation

For purposes of illustration, simulated sinograms were utilized in this study instead of raw data obtained by commercial CT scanners. To generate simulated sinograms, the pixel values of CT images stored in a DICOM file were first converted into the corresponding linear attenuation coefficients (LACs), and any negative LAC due to random noise was changed to zero. To investigate the effects of number of projection views and detector size on the model performance, three sets of sinograms were generated based on the LAC images. Specifically, different projection data were utilized to generate sinograms and reconstruction images for the comparative study. First, sinograms with 360 projection views over 180 degrees and 729 detectors, Sino360×729, (full data) were computed using the 2D parallel-beam Radon transform. Sino360×729 were then used to produce sparser sinograms by uniformly subsampling projection views (in horizontal direction) and averaging projection data from adjacent detectors (in vertical direction). Sino120×240 with 120 projection views and 240 detectors (limited data) and Sino40×80 with 40 projection views and 80 detectors (sparse data) were created by downsampling and averaging Sino360×729 by factors of 3 and 9, respectively (FIG. 4). All sinograms were resized to create standardized 360×729 images.

### Image Reconstruction

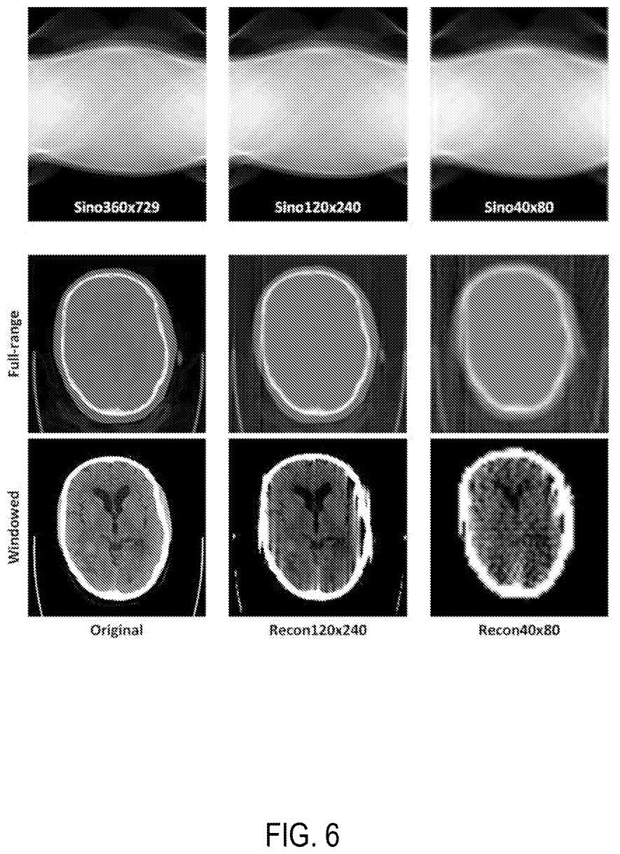

Reconstructed images were generated to compare with models obtained using the corresponding three sets of sinograms. For Sino360×729, original CT images were used as the reconstructed images, and full-view sinogram were obtained using a commonly-used analytical filtered back projection (FBP) algorithm, also known as inverse Radon transform. For sparser sinograms, however, other complex algorithms were needed to produce high-quality image reconstructions. For this study, a deep learning approach was used, implementing a ReconUnet, a modified version of an U-Net with residual learning, to take FPP images as input and create corresponding reconstructed images of high quality. This approach was based on previous work demonstrating that the deep learning can compare favorably to state-of-the-art iterative algorithms for sparse-view image reconstruction. The best ReconUnet models were deployed on Sino120×240 and Sino40×80 to obtain corresponding reconstructed images, such as Recon120×240 and Recon40×80 (FIG. 6). Root mean square error (RMSE) values between original CT images and reconstructed images were 16 times smaller when using ReconUnet as compared FBP for Sino120×240, and 7 times smaller for Sino40×80.

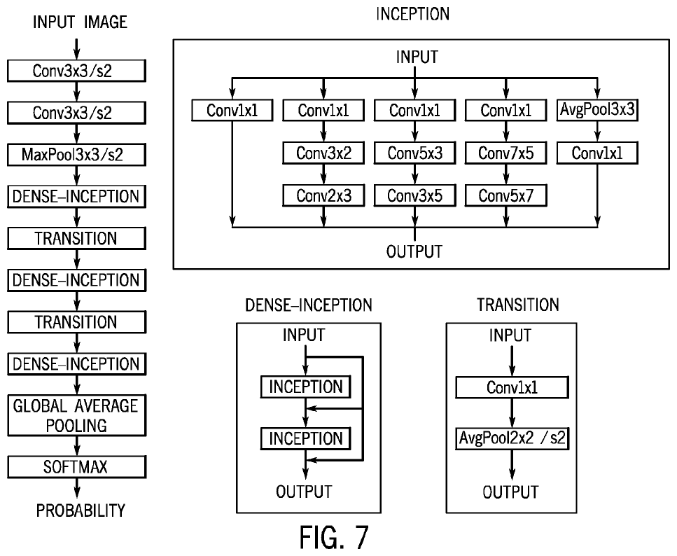

SinoNet was designed for analyzing sinograms through inspiration of Inception modules with multiple convolutional and pooling layers and dense connection for efficient use of model parameters. As illustrated in FIG. 7, an Inception module was modified by using various sized rectangular convolutional filters including height-wise and width-wise filters for specializing in extracting projection view and detector dominant features from sinusoidal curves, respectively. Each Dense-Inception block contained two densely connected Inception modules, followed by a Transition block to reduce the number and size of feature maps for computational efficiency, as suggested in the original literature. As shown in FIG. 7, the modified Inception module contained multiple rectangular convolution filters of various size: height-wise rectangular filters (projection view dominant) in red; width-wise rectangular filters (detector dominant) in light orange; where “Conv3×3/s2” indicates a convolutional layer with 3×3 filters and 2 stride, “Conv3×2” indicates a convolution layer with 3×2 filters and a stride; Conv=convolution layer, MaxPool=max pooling layer, AvgPool=average pooling layer

### Performance Evaluation and Statistical Analysis

Accuracy was used as performance metric for comparing the body part recognition models, and Receiver operating characteristic (ROC) and area under the ROC curve (AUC) were utilized for evaluating the models to predict presence of ICH. All the performance metrics were calculated using a machine learning library available in python 2.7.12. To compute 95% confidence intervals (CIs) of the metrics for assessment of statistical significance, a non-parametric bootstrap approach was employed.

### Network Training

All deep learning models for classification were trained for 30 epochs using a mini-batch stochastic gradient descent with Nesterov momentum of 0.9, batch size of 64, weight decay of 5×10⁻⁵. The base learning rate 0.005 was decayed by a factor of 10 every 10 epochs to ensure a stable convergence of training cost function. ReconUnet, a deep learning model for image reconstruction, was trained for 100 epochs using Adam optimization algorithm with default settings and a base learning rate of 0.001. The best models for classification were selected based on validation losses, and the best ReconUnet models were chosen based on validation RMSE values. For the ICH detection task, Inception-v3 and SinoNet pre-trained on training dataset of body part recognition were utilized when using reconstructed images and sinograms, in order to make a fair comparison.

### Infrastructure

Radon and iradon functions were implemented in Matlab 2018a to generate sinograms from original CT images and obtain FBP reconstructions from sinograms, respectively. For experiments associated with deep learning, Keras (version 2.1.1) with a Tensorflow backend (version 1.3.0) was used to implement deep learning models, and experiments were performed using an NVIDIA Devbox (Santa Clara, Calif.) equipped with four TITAN X GPUs with 12 GB of memory per GPU.

### Baseline Settings

For original CT images, images with a full range of HU values (Full-range) were utilized. In addition, images with window levels (WL) and window widths (WW) predefined for target application were utilized as follows: abdomen window (WL=40 HU, WW=400 HU) for body part recognition and brain window (WL=50 HU, WW=100 HU) for ICH detection. Inception-v3 was selected as a competitive comparison to SinoNet for analyzing sinograms because Sinonet architecture was inspired by Inception-v3, which demonstrated its performance of image classification in the ImageNet Large Scale Visual Recognition Challenge (ILSVRC). Inception-v3 was altered by removing the last fully-connected layers and attaching a global average pooling (GAP) layer, a fully-connected layer, and a softmax layer with outputs of the same number of categories: 16 outputs for body part recognition and 2 outputs for ICH detection.

### Results

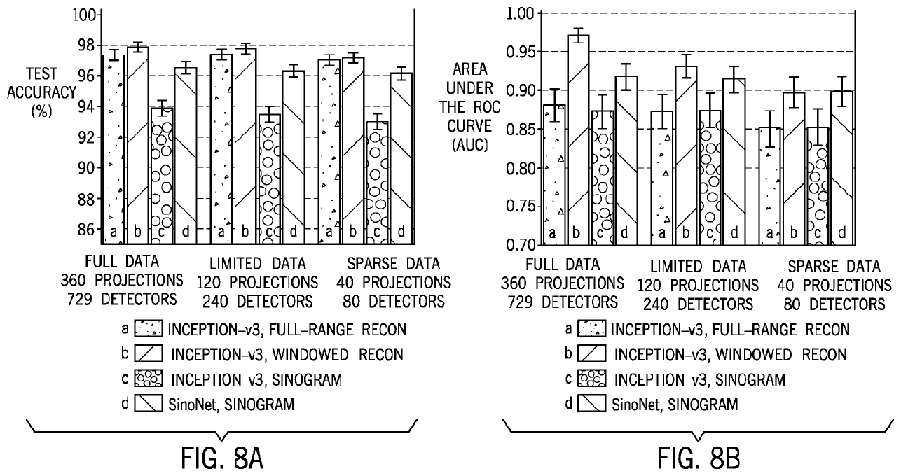

Reconstructed CT images having a full range of HU values without window settings and windowed images were utilized. Inception-v3 convolutional neural network (CNN) was used as baseline network and a customized CNN, SinoNet was developed for an efficient analysis of sinograms using multiple rectangular convolution filters with densely connected layers. Results of the systematic study for the two different tasks are shown FIGS. 8A and 8B. For body part recognition, SinoNet with sinograms achieved test accuracies of 96.6% (95% CI, 96.2%-97.0%), 96.3% (95% CI, 95.9%-96.7%), and 96.2% (95% CI, 95.8%-96.6%) for Full, Limited, and Sparse data, respectively. These represent much better results compared to those of Inception-v3 using sinograms, producing only about 1% lower than the performance achieved when using full-range and windowed reconstruction images (FIG. 8A). For ICH detection, SinoNet with sinograms achieved AUCs of 0.918 (95% CI, 0.900-0.934), 0.915 (95% CI, 0.897-0.931), and 0.899 (95% CI, 0.879-0.917) for Full, Limited, and Sparse data, respectively, which are significantly higher than ones of the baseline model using corresponding full-range CT images and sinograms (FIG. 8B). The performance of SinoNet with Sino360×729 was 0.054 lower than the AUC of Inception-v3 using windowed reconstruction images from the full data, while the performance was comparable to the one when using windowed reconstruction images from the sparse data.

### Discussion

In this study, the feasibility of using machine learning for recognition and diagnosis/screening tasks directly using medical raw data (i.e. without image reconstruction) was demonstrated. Specifically, results show that CT sinogram-based body part recognition achieved accuracy of about 96% that was close to the performance of CT-image based approach, irrespective of the radiation dose. For the ICH detection task, the accuracy of the sinogram model was at least 90% for all 3 scanning geometries. These results demonstrate the potential benefit of a sinogram based approach in emergency medical services where saving time (e.g. by eliminting image reconstruction) is critical. Also, the performance of the present sinogram-based model was comparable with the CT image model when the projection data were collected from sparse projection views and/or large detector size. This allows the present approach to be utilized in situations where low-dose or low-cost CTs are needed.

As described above, sinograms used in this study were were simulated by applying the 2D parallel-beam Radon transform to the reconstructed CT images. A more realistic simulation could apply cone-beam scanning geometry to generate projection data. Also, Poisson noise could be added in the simulated data. In addition, although the sinogram based model achieved detection accuracy above 90%, there may still be a gap between the performance of the sinogram model and the CT image model. Therefore, a number of improvements for enhancing performance and circumventing any limitations of the sinogram-based approach are envisioned. For instance, in some implementations, the present sinogram-based method may be combined with the CT image method to reduce error rates associated with each individual method. Also, the present sinogram-based could be used as first-line screening or triage, while the CT image method could be used to confirm such first-line interpretation and localize the conditions.

As appreciated from discussion above, the present approach provides a number of advantages and addresses shortcomings of previous technologies. Specifically, the present approach allows for cheaper and simpler CT scanners, and less radiation dose to patients. This is because the present approach can utilize limited or sparse data and produce acceptable, and in some cases, enhanced results. Therefore, utilizing fewer projection views results in less radiation dose received by a patient. Also, fewer detectors reduce space, cost and complexity requirements for a scanner.

The present invention has been described in terms of one or more preferred embodiments, and it should be appreciated that many equivalents, alternatives, variations, and modifications, aside from those expressly stated, are possible and within the scope of the invention.

What is claimed is:

1. A method for analyzing medical image data using a computing device, the method comprising:
using an input on the computing device to receive image data acquired from a subject, wherein the image data is in a raw data domain;
applying, using the computing device, a trained machine learning algorithm to the image data, wherein the trained machine learning algorithm is configured to perform a predetermined analysis on the image data; and
using the computing device, generating a report indicative of the predetermined analysis.

2. The method of claim 1, wherein the image data comprises computed tomography (CT) data, magnetic resonance (MR) data, single-photon emission computed tomography (CT) data, positron emission tomography (PET) data, ultrasound (US) data, or a combination thereof.

3. The method of claim 1, wherein the raw data domain comprises a sinogram domain.

4. The method of claim 1, wherein the method further comprises generating the trained machine learning algorithm by using image data in the raw data domain obtained from a plurality of subjects.

5. The method of claim 1, wherein the predetermined analysis comprises identifying at least one target in the image data.

6. The method of claim 5, wherein the at least one target comprises a benign tumor tissue, a malignant tumor tissue, or a hemorrhage.

7. The method of 5, wherein the at least one target comprises a tissue, an anatomical structure, or an organ.

8. The method of claim 5, wherein the method further comprises classifying the at least one target identified in the image data.

9. A system for analyzing medical imaging data, the system comprising:
an input in communication with an image data source and configured to receive image data therefrom;
at least one processing unit configured to:
receive, from the input ,image data acquired from a subject;
apply a trained machine learning algorithm to the image data, wherein the trained machine learning algorithm is configured to perform a predetermined analysis on the image data; and
generate a report indicative of the predetermined analysis; and
an output configured to provide the report.

10. The system of claim 9, wherein the image data source is an imaging system.

11. The system of claim 9, wherein the input is configured to receive image data comprising computed tomography (CT) data, magnetic resonance (MR) data, single-photon emission computed tomography (CT) data, positron emission tomography (PET) data, ultrasound (US) data, or a combination thereof.

12. The system of claim 9, wherein the raw data domain comprises a sinogram domain.

13. The system of claim 9, wherein the at least one processing unit is further configured to generate the trained machine learning algorithm by using image data in the raw data domain obtained from a plurality of subjects.

14. The system of claim 9, wherein the predetermined analysis comprises identifying at least one target in the image data.

15. The system of claim 14, wherein the at least one processing unit is configured to separate image data associated with the at least one target.

16. The system of claim 14, wherein the at least one target comprises a benign tumor tissue, a malignant tumor tissue, or a hemorrhage.

17. The system of claim 14, wherein the at least one target comprises a tissue, an anatomical structure, or an organ.

18. The system of claim 14, wherein the at least one processing unit is further configured to classify the at least one target identified in the image data.
